(19) United States
(12) Patent Application Publication
(10) Pub. No.: US 2011/0184321 A1
(43) Pub. Date: Jul. 28, 2011

(54) HARD TO REACH FRACTURE APPLICATOR STRAP

(75) Inventors: Nicholas Scott Reback, Monona, WI (US); Kevin Alan Williams, Madison, WI (US); Craig Patrick Conner, Madison, WI (US); Mark W. Cors, Columbus, TN (US); Jesse Charles Darley, Madison, WI (US); Michael John Guse, Marshall, WI (US); Stephen Andrew Latham, Sun Prairie, WI (US); Jason Splan, McFarland, WI (US)

(73) Assignee: Smith & Nephew, Inc., Memphis, TN (US)

(21) Appl. No.: 12/918,098

(22) PCT Filed: Feb. 27, 2009
(86) PCT No.: PCT/US09/35476

§ 371 Date: Feb. 1, 2011

Related U.S. Application Data
(60) Provisional application No. 61/032,692, filed on Feb. 29, 2008.
(60) Provisional application No. 61/086,900, filed on Aug. 7, 2008.

(51) Int. Cl. A61N 7/00

(52) U.S. Cl. 601/2

(57) ABSTRACT

There may be provided a hard-to-reach fracture applicator strap assembly (10). The assembly (10) includes a bottom portion (12); a stiffener (20) in contact with the bottom portion (12); a large pouch portion (16) in contact with the stiffener (20); a small pouch portion (18) in contact with the stiffener (20); and a top portion (14) in contact with at least one of the large pouch portion (16) and the small pouch portion (18).

### CROSS-REFERENCE TO RELATED APPLICATIONS

This application claims the benefit of U.S. Provisional Application No. 61/032,692, filed Feb. 29, 2008 and U.S. Provisional Application No. 61/086,900, filed Aug. 7, 2008. The disclosure of each application is incorporated by reference in its entirety.

### BACKGROUND OF THE INVENTION

1. Field of the Invention

Embodiments of the present invention generally relate to ultrasound devices and more particularly to application of ultrasound energy in hard to reach places.

2. Related Art

There is difficulty in applying ultrasound devices to certain areas of the body. These areas have been identified as, but are not limited to, the clavicle, the proximal femur, the pelvis, the fingers, and the toes.

There have been three solutions to this problem in the past: (1) taping an ultrasound applicator to the body; (2) a combination of straps to hold the applicator against the body; or (3) having the patient hold the device in place. The problem with tape is the adhesive may negatively interact with the skin and cause irritation. The combination of straps poses difficulty in application by a patient without a caregiver. Holding the device limits mobility and activity during treatment, as well as causing some discomfort by the patient.

### SUMMARY OF THE INVENTION

According to some aspects of the present invention there may be provided a hard-to-reach fracture applicator strap assembly. The assembly may include a bottom portion; a stiffener in contact with the bottom portion; a large pouch portion in contact with the stiffener; a small pouch portion in contact with the stiffener; and a top portion in contact with at least one of the large pouch portion and the small pouch portion.

According to some embodiments of the present invention, the bottom portion and the top portion may have a shape selected from the group consisting of bow-tie, oval, rectangular, elliptical, lozenge, navicular, oblong, and triangular.

According to some embodiments of the present invention, the bottom portion and the top portion are made of a material selected from the group consisting of polymers, synthetic fabrics, and natural fabrics.

According to some embodiments of the present invention, the assembly may further include edging and stitching.

According to some embodiments of the present invention, the large pouch portion and the small pouch portion are arranged to provide a space D around a perimeter of the stiffener, and the space D may range from about 0.1 inches to about 1 inch.

According to some embodiments of the present invention, the large pouch portion and the small pouch portion each have one or more weighted compartments.

According to some embodiments of the present invention, the weighted compartments each further comprise a weight selected from the group of metal pellets and plastic pellets.

According to some embodiments of the present invention, the large pouch portion has four compartments and the small pouch portion has two compartments.

According to some embodiments of the present invention, the bottom portion, the stiffener, and the top portion each include an opening. The openings may be co-axial when assembled. The openings may be shaped and dimensioned to receive a retaining and alignment fixture.

In some embodiments, the invention further includes a retaining and alignment fixture and a transducer mounted within the retaining and alignment fixture.

In some embodiments, the number and length of the legs could be modified to provide the same function.

In some embodiments, the material on the bottom of the device could be changed to increase the adhesive nature of the device.

In some embodiments, adhesive material could be added to the device to attach it to the skin for added security.

In some embodiments, hook-and-loop tape material could be added to the device to attach straps for added security.

In some embodiments, adjustable positioning of strap attachment could be added to the device for flexibility of positioning.

The invention may include one or more of the following advantages: (1) weight and weight placement provide security in the placement without posing any skin interaction; (2) an opening allows for transmission of ultrasound waves; (3) a non-slip surface on a bottom of the applicator may be provided to reduce slippage; (4) a longer leg on one end of the device may allow it to sit evenly over the patient's shoulder for treatment of the clavicle; and (5) a side curvature may allow for positioning in crevices of the body or along the neck area. The invention may allow for hands-free use, application without adhesive, and the ability to apply without assistance.

Further areas of applicability of the invention will become apparent from the detailed description provided hereinafter. It should be understood that the detailed description and specific examples, while indicating the particular embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 illustrates a hard-to-reach (HTR) fracture applicator strap assembly in a first embodiment.

FIG. 2 illustrates a bottom portion of the HTR fracture applicator strap assembly.

FIG. 3 illustrates a top portion of the HTR fracture applicator strap assembly.

FIG. 4 illustrates a large pouch portion of the HTR fracture applicator strap assembly.

FIG. 5 illustrates a small pouch portion of the HTR fracture applicator strap assembly.

FIG. 6 illustrates a stiffener portion of the HTR fracture applicator strap assembly.

FIG. 7 illustrates the first embodiment in use.

FIG. 8 illustrates the first embodiment and a retaining and alignment fixture disassembled.

FIG. 9 illustrates the first embodiment and the retaining and alignment fixture assembled.

FIG. 10A illustrates the first embodiment in a top view.

FIG. 10B is a sectional side view along lines A-A in FIG. 10A.

FIG. 10C is a detailed view of the area B in FIG. 10B.

FIG. 11 illustrates a second embodiment of the of the HTR fracture applicator strap assembly.

FIG. 12 illustrates a third embodiment of the of the HTR fracture applicator strap assembly.

FIG. 13 illustrates a fourth embodiment of the of the HTR fracture applicator strap assembly.

FIG. 14 illustrates a fifth embodiment of the of the HTR fracture applicator strap assembly.

FIG. 15 illustrates a sixth embodiment of the of the HTR fracture applicator strap assembly.

FIG. 16 illustrates a seventh embodiment of the of the HTR fracture applicator strap assembly.

FIG. 17 illustrates an eighth embodiment of the of the HTR fracture applicator strap assembly.

FIG. 18 illustrates a ninth embodiment of the of the HTR fracture applicator strap assembly.

### DETAILED DESCRIPTION OF THE EMBODIMENTS

The following description of the depicted embodiment(s) is merely exemplary in nature and is in no way intended to limit the invention, its application, or uses.

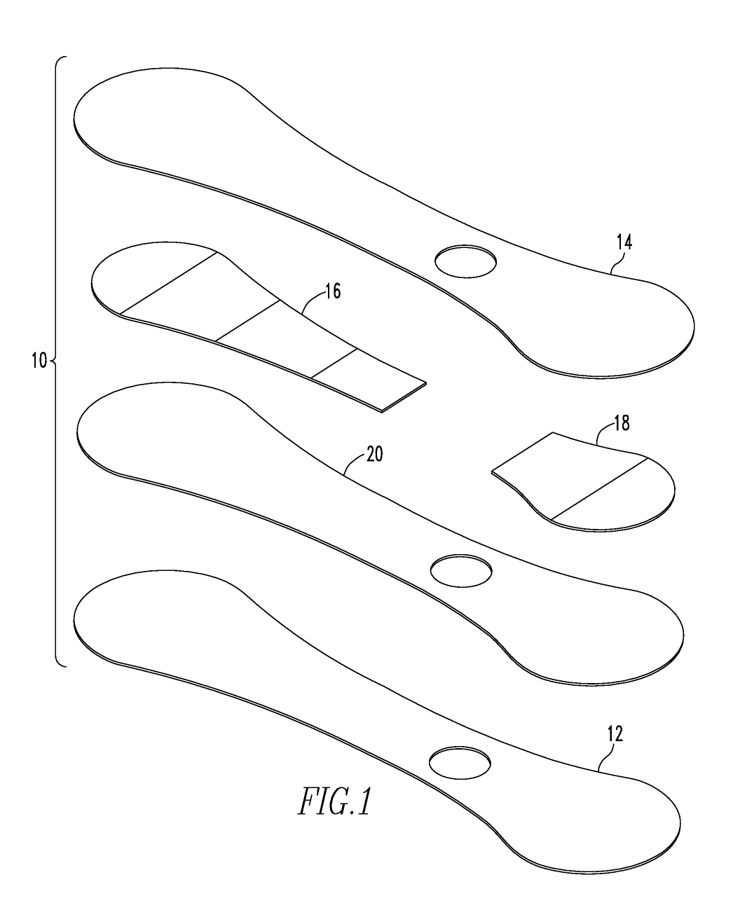

FIG. 1 illustrates a hard-to-reach (HTR) fracture applicator strap assembly 10 in a first embodiment. The HTR fracture applicator strap assembly 10 may include a bottom portion 12, a top portion 14, a large pouch portion 16, a small pouch portion 18, and a stiffener 20. In some embodiments, these components may be glued, fastened, or sewn together. The HTR fracture applicator strap assembly 10 may be any number of shapes, including, but not limited to, oval, rectangular, elliptical, lozenge, navicular, oblong, or triangular. In the depicted embodiment, the HTR fracture applicator strap assembly 10 has a bow-tie shape. The bow-tie shape may aid in holding the HTR fracture applicator strap assembly 10 in place and allow it to be placed close to the neck or desired location of the neck area. The HTR fracture applicator strap assembly 10 is flexible and generally conforms to the surface onto which it is placed. In some embodiments, the HTR fracture applicator strap assembly 10 may be machine washable.

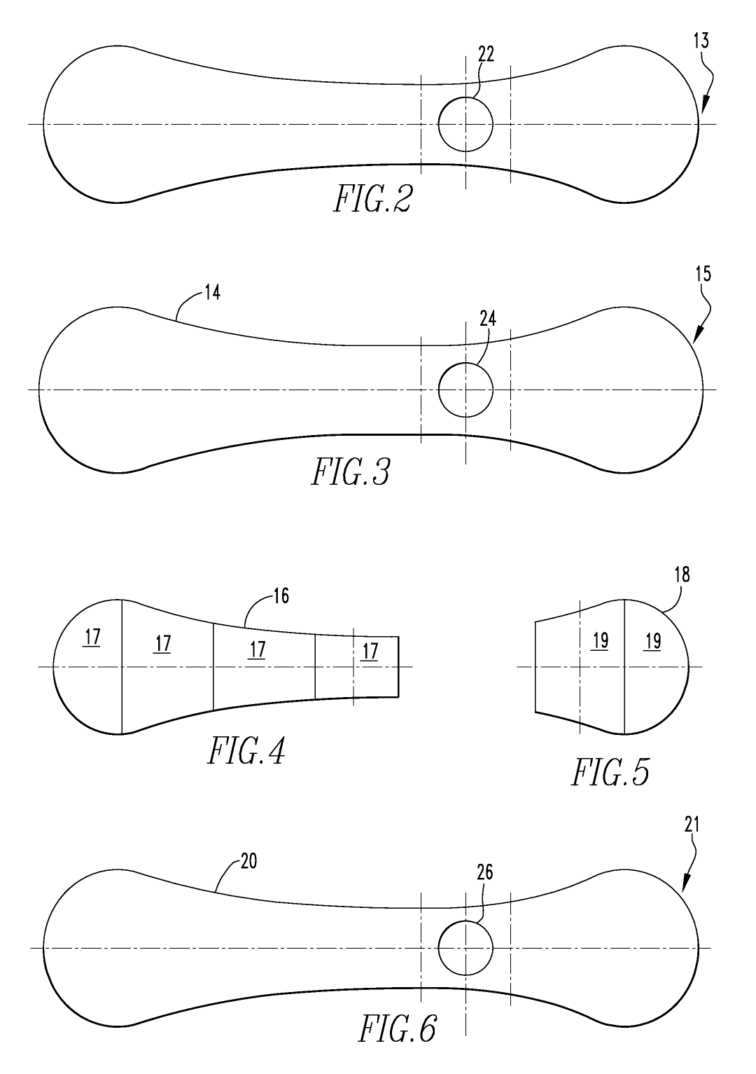

FIG. 2 illustrates the bottom portion 12 of the HTR fracture applicator strap assembly 10. The bottom portion 12 contacts the user's skin and so it is desirable, but not necessary, that the bottom portion 12 be made of a material that does not cause skin irritation. The bottom portion 12 may be made of any number of materials, such as polymers, synthetic fabrics, or natural fabrics. In the depicted embodiment, the bottom portion 12 is made of a PVC coated polyester knit available from Eastex Products, Inc., 275 Centre Street, Building 6, Holbrook, Mass. In some embodiments, the bottom portion 12 may be textured on one side to reduce slippage. The bottom portion 12 may be any number of shapes, including, but not limited to, oval, rectangular, elliptical, lozenge, navicular, oblong, or triangular. In the depicted embodiment, the bottom portion 12 has bow-tie shape. The shape of the bottom portion 12 may aid in holding the HTR fracture applicator strap assembly 10 in place.

The bottom portion includes an opening 22. The opening 22 may be located in the center of the bottom portion 12 or offset. In the depicted embodiment, the bottom portion 12 is about 14.5 inches long and about 3.5 inches wide. Of course other dimensions may be used, and several sizes may be available to accommodate both pediatric and adult patients. In the embodiment shown in FIG. 2, the opening 22 is offset and is located about 5.2 inches from an end 13.

FIG. 3 illustrates the top portion 14 of the HTR fracture applicator strap assembly 10. The top portion 14 may be made of any number of materials, such as polymers, synthetic fabrics, or natural fabrics. In the depicted embodiment, the top portion 14 is made of polyurethane coated polyester knit available from Eastex Products, Inc. The top portion 14 covers the large pouch portion 16, the small pouch portion 18, and the stiffener portion 20. The top portion 14 may stretch as the HTR fracture applicator strap assembly 10 generally conforms to a surface. The top portion 14 may be any number of shapes, including, but not limited to, oval, rectangular, elliptical, lozenge, navicular, oblong, or triangular. In the depicted embodiment, the top portion 14 has bow-tie shape. The shape of the top portion 14 may aid in holding the HTR fracture applicator strap assembly 10 in place.

The top portion 14 includes an opening 24. The opening 24 may be located in the center of the top portion 14 or offset. The top portion 14 may be longer and/or wider than the bottom portion 12 in some embodiments. In the depicted embodiment, the top portion 14 is about 14.7 inches long and about 3.7 inches wide. Of course other dimensions may be used, and several sizes may be available to accommodate both pediatric and adult patients. In the embodiment shown in FIG. 3, the opening 24 is offset and is located about 5.2 inches from an end 15.

FIG. 4 illustrates the large pouch portion 16 of the HTR fracture applicator strap assembly 10. The large pouch portion 16 is a weighted pouch with one or more compartments 17. The compartments 17 help keep the HTR fracture applicator strap assembly 10 in place and may aid in keeping the bottom portion 12 in contact with the user's skin. In the depicted embodiment, the large pouch portion 16 includes four compartments, but any number of compartments may be used. As examples, the large pouch portion 16 may have two, three, five, or six compartments. The compartments 17 are formed by segmenting the large pouch portion 16. As examples, the large pouch portion 16 may be segmented by plastic welding, glue, or by thread.

Many different materials may be used to provide weight, including metal or plastic pellets. In the depicted embodiment, each compartment 17 contains number 7 steel shot, with about 422 shots per ounce, and the large pouch portion 16 contains a total of about 10.5 ounces. Of course, smaller or larger shot may be used, and the large pouch portion 16 may contain a greater or smaller amount of shot. As an example, the large pouch portion 16 may contain a weight in the range of about 7 ounces to about 16 ounces. In the depicted embodiment, the large pouch portion 16 is about 7.6 inches long and about 3.0 inches wide. Of course other dimensions may be used, and several sizes may be available to accommodate both pediatric and adult patients.

FIG. 5 illustrates the small pouch portion 18 of the HTR fracture applicator strap assembly 10. The small pouch portion 18 is a weighted pouch with one or more compartments 19. The compartments 19 help keep the HTR fracture applicator strap assembly 10 in place and may aid in keeping the bottom portion 12 in contact with the user's skin. In the depicted embodiment, the small pouch portion 18 includes two compartments 19, but any number of compartments may be used. As examples, the small pouch portion 18 may have one, three, four, five, or six compartments. The compartments 19 are formed by segmenting the small pouch portion 18. As examples, the small pouch portion 18 may be segmented by plastic welding, glue, or by thread.

Many different materials may be used to provide weight, including metal or plastic pellets. In the depicted embodiment, each compartment 19 contains number 7 steel shot, with about 422 shots per ounce, and the small pouch portion 18 contains a total of about 10.5 ounces. Of course, smaller or larger shot may be used, and the small pouch portion 18 may contain a greater or smaller amount of shot. As an example, the small pouch portion 18 may contain a weight in the range of about 7 ounces to about 16 ounces. In the depicted embodiment, the small pouch portion 18 is about 3.4 inches long and about 3.0 inches wide. Of course other dimensions may be used, and several sizes may be available to accommodate both pediatric and adult patients.

The large pouch portion 16 and the small pouch portion 18 may be of many different materials. In the depicted embodiment, the pouches 16, 18 are made of a polymer, such as polyvinyl, and has a thickness of about four mil (0.004) inches. The pouches 16, 18 may be made of a polymer with a thickness of about two mil to about eight mil.

FIG. 6 illustrates the stiffener portion 20 of the HTR fracture applicator strap assembly 10. The stiffener portion 20 is provided to reduce the overall flexibility of the HTR fracture applicator strap assembly 10. The stiffener portion 20 may be made of many different materials, but in the depicted embodiment, the stiffener portion 20 is made of 1200 denier ballistic nylon. The stiffener portion 20 includes an opening 26. The opening 26 may be located in the center of the stiffener portion 20 or offset. The stiffener portion may be generally shorter and/or narrower than the bottom portion 12 in some embodiments. In the depicted embodiment, the stiffener portion 20 is about 14.5 inches long and about 3.5 inches wide. Of course other dimensions may be used, and several sizes may be available to accommodate both pediatric and adult patients. In the embodiment shown in FIG. 6, the opening 26 is offset and is located about 5.2 inches from an end 21. The stiffener portion 20 may be any number of shapes, including, but not limited to, oval, rectangular, elliptical, lozenge, navicular, oblong, or triangular. In the depicted embodiment, the stiffener portion 20 has bow-tie shape to match the bottom portion 12. In some embodiments, the shape of the stiffener may be different than the shape of the bottom portion 12.

In the depicted embodiments, openings 22, 24, 26 are all substantially co-axial. In some embodiments, the openings 22, 24, 26 are not co-axial but do overlap in diameter.

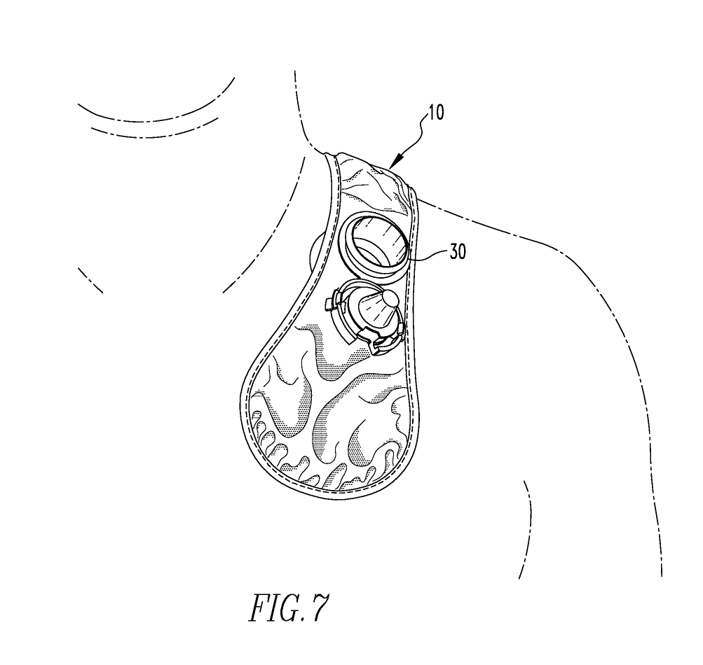

FIG. 7 illustrates the first embodiment of the HTR fracture applicator strap assembly 10 in use for applying ultrasound energy to a clavicle. In the depicted embodiment, the HTR fracture applicator strap assembly 10 is flexible and contours itself to the area near the clavicle. A retaining and alignment fixture 30 is attached to the HTR fracture applicator strap assembly 10. The retaining and alignment fixture 30 is adapted to hold an ultrasound energy transducer (not shown). In the depicted embodiment, a larger portion of the HTR fracture applicator strap assembly 10 extends over the back of the user than in the front. The user can easily move the HTR fracture applicator strap assembly 10 to adjust for comfort.

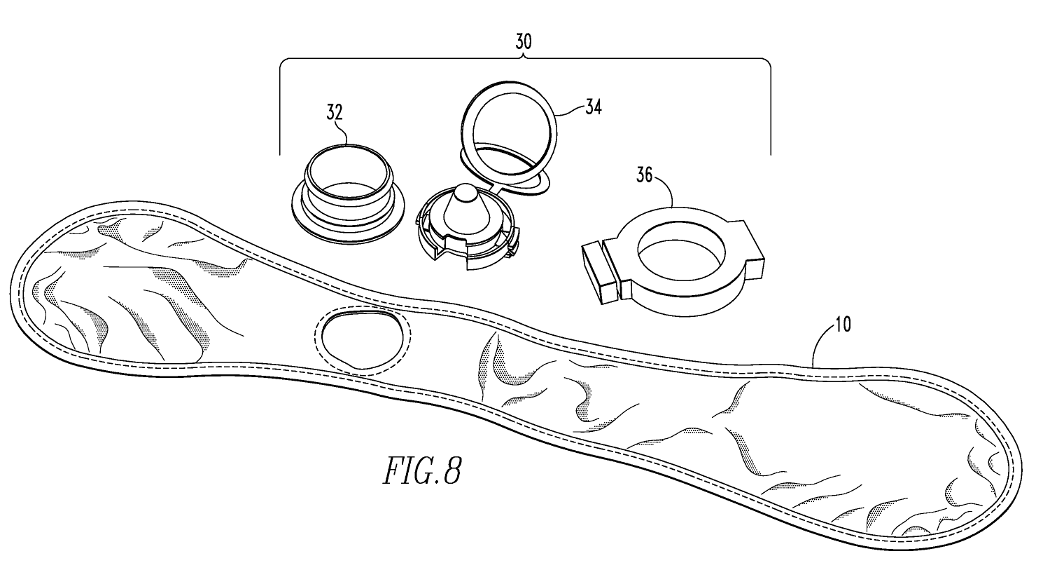

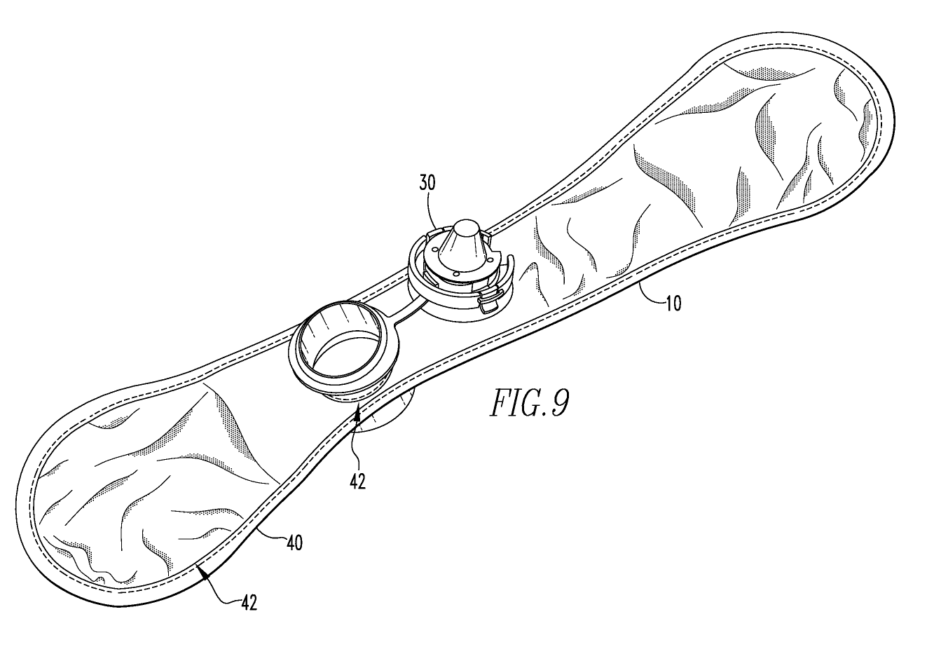

FIG. 8 illustrates the first embodiment of the HTR fracture applicator strap assembly 10 and a retaining and alignment fixture 30 disassembled. The retaining and alignment fixture 30 may include an insert 32, a housing 34, and a spacer 36. In the depicted embodiment, the insert 32 includes an axial bore and a substantially circular periphery to mount the ultrasound transducer. The housing 34 may be inserted into an upper portion of the insert 32 to enclose the components therein. The spacer 36 maintains the insert 32 at a predetermined distance from a user's skin to prevent window edema or similar injury.

FIG. 9 illustrates the first embodiment of the HTR fracture applicator strap assembly 10 and the retaining and alignment fixture 30 assembled. In the embodiment depicted in FIG. 9, the HTR fracture applicator strap assembly 10 includes an edging 40 and stitching 42. The edging 40 is made of polyurethane coated polyester knit available from Eastex Products but other materials could equally be used. In the depicted embodiment, the edging 40 is about 0.5 inches wide but other sizes could equally be used. The stitching 42 is made from industrial thread and may be made from natural or synthetic fibers.

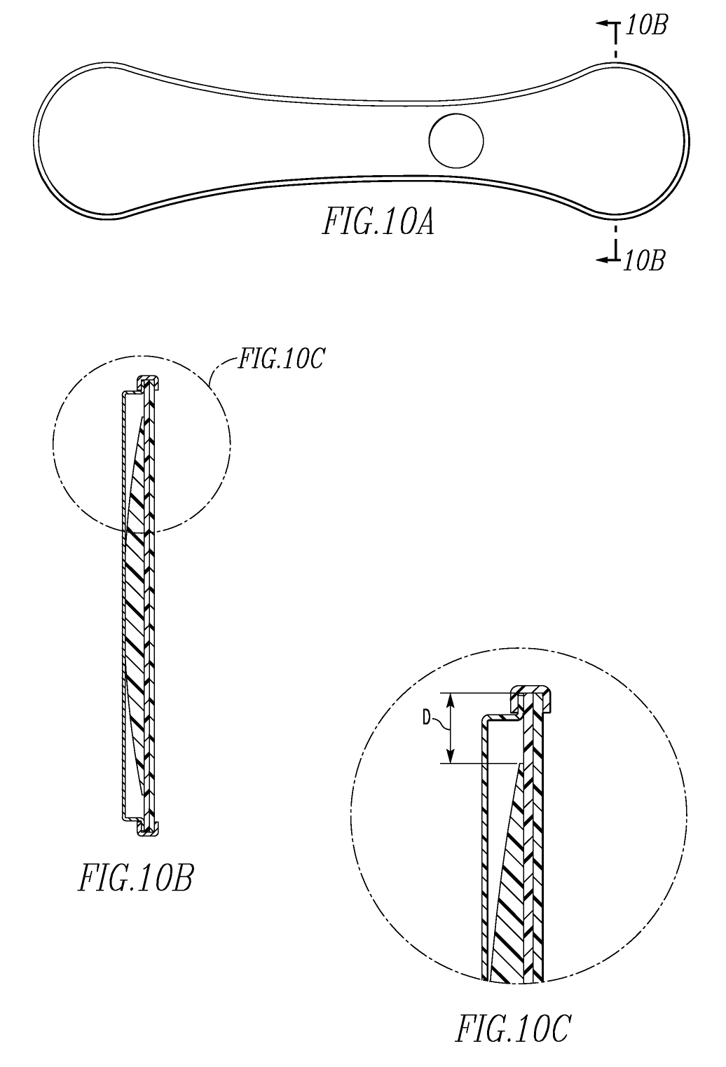

FIGS. 10A-10C illustrate the first embodiment of the HTR fracture applicator strap assembly 10. As best seen in FIG. 10C, the pouches 16, 18 may be arranged to provide a space D around the perimeter. The space D may range from about 0.1 inches to about 1 inch. In the depicted embodiment, the space D is about 0.25 inches.

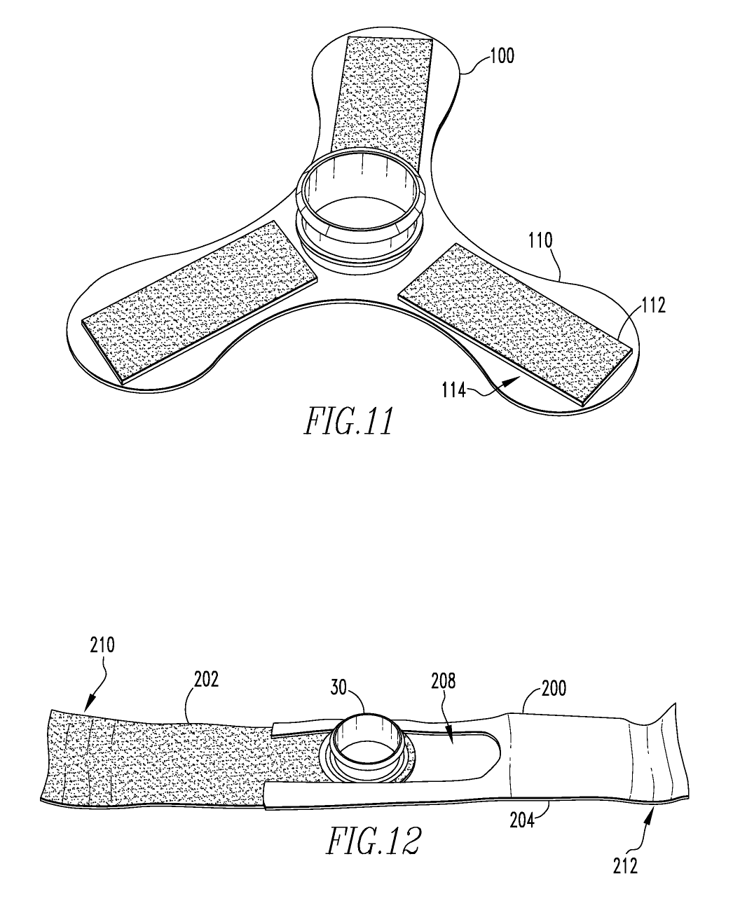

FIG. 11 illustrates a second embodiment of the of the HTR fracture applicator strap assembly 100. The second embodiment is a three-legged applicator. In the depicted embodiment, each leg 110 is evenly spaced apart. Each leg 110 may include a patch 112. The patch 112 may be formed of gel adhesive, hydrogel, pressure sensitive adhesive, or one-side of hook-and-loop tape. The patch 112 may be located on a top 114, bottom (not shown), or both the top and the bottom. The gel adhesive or hydrogel may be used to frictionally engage the user's skin to help hold the HTR fracture applicator strap assembly 100 in place. If the patch 112 is made of one side of hook-and-loop tape, a strap (not shown) having the other side of the hook-and-loop tape may be used to hold the HTR fracture applicator strap assembly 100 in place.

FIG. 12 illustrates a third embodiment of the of the HTR fracture applicator strap assembly 200. The third embodiment is a variable length, weighted version with weight located just at the ends 210, 212. The HTR fracture applicator strap assembly 200 includes a first portion 202 and a second portion 204. The portions 202, 204 overlap, and the second portion 204 has a u-shaped cutout 208 to accommodate the retaining and alignment fixture 30. The portions 202, 204 may be connected using hook-and-loop tape.

FIG. 13 illustrates a fourth embodiment of the of the HTR fracture applicator strap assembly 300. The fourth embodiment is a dumbbell shaped version. The fourth embodiment may have adhesive on a top side or bottom side, hook-and-loop tape on a top side or bottom side, or a combination of adhesive and hook-and-loop tape applicator. In some embodiments, gel, hydrogel, or pressure sensitive adhesive may be used. A strap may be used to aid in holding the HTR fracture applicator strap assembly 300 in place. The HTR fracture applicator strap assembly 300 also includes an opening 310 to receive the retaining and alignment fixture 30. The opening 310 may be centered or offset between ends 314, 316.

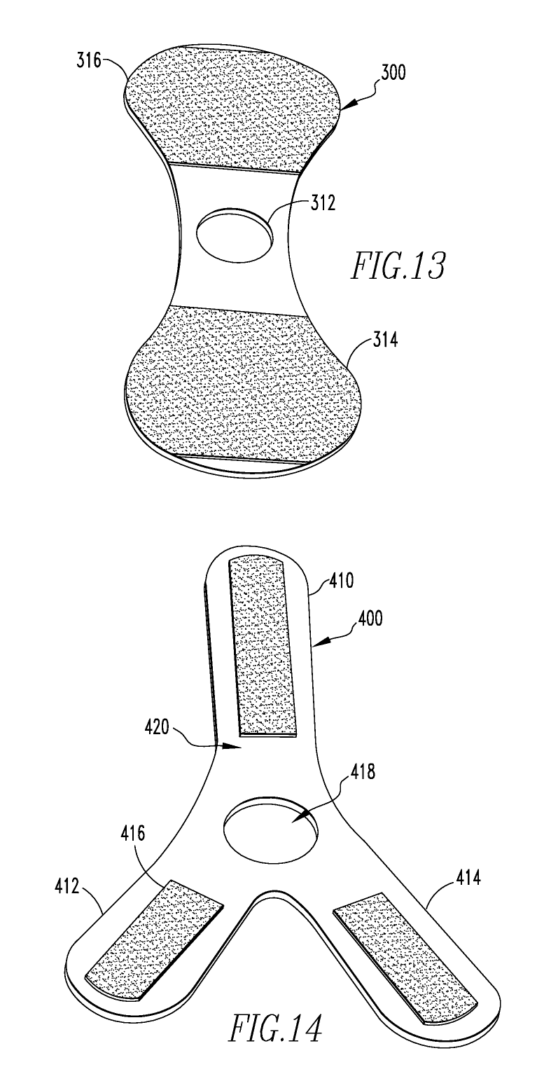

FIG. 14 illustrates a fifth embodiment of the of the HTR fracture applicator strap assembly. The fifth embodiment is a three-legged version with uneven leg spacing and/or length. In the depicted embodiment, legs 412, 414 have an acute spacing but leg 410 is at an obtuse angle relative to the other legs 412, 414. Legs 410, 412, 414 may all have the same length or different lengths. In the depicted embodiment, leg 410 is longer than legs 412, 414. Each leg 410, 412, 414 may include a patch 416. The patch 416 may be formed of gel adhesive, pressure sensitive adhesive, hydrogel, or one-side of hook-and-loop tape. The patch 416 may be located on a top 420, bottom (not shown), or both the top and the bottom. The gel adhesive, pressure sensitive adhesive, or hydrogel may be used to frictionally engage the user's skin to help hold the HTR fracture applicator strap assembly 400 in place. If the patch 416 is made of one side of hook-and-loop tape, a strap (not shown) having the other side of the hook-and-loop tape may be used to hold the HTR fracture applicator strap assembly 400 in place. The HTR fracture applicator strap assembly 400 may include an opening 418.

FIG. 15 illustrates a sixth embodiment of the of the HTR fracture applicator strap assembly. The sixth embodiment is a four-legged, symmetrical, non-weighted applicator. The sixth embodiment may have adhesive on one side, hook-and-loop tape on one side, or a combination of adhesive and hook-and-loop tape applicator without weight. In some embodiments, gel, hydrogel, or pressure sensitive adhesive may be used.

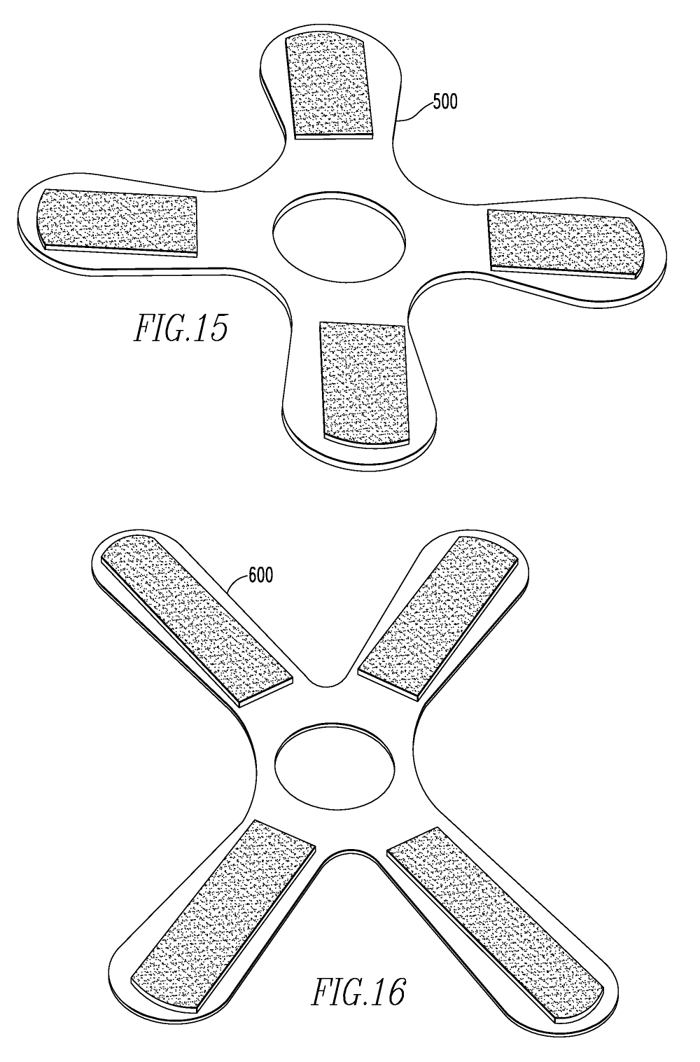

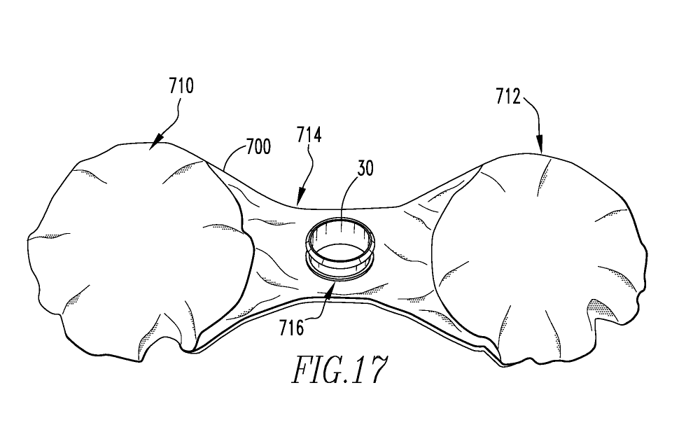

FIG. 16 illustrates a seventh embodiment of the of the HTR fracture applicator strap assembly. The seventh embodiment is a four-legged uneven non-weighted applicator. The seventh embodiment may have adhesive on one side, hook-and-loop tape on one side, or a combination of adhesive and hook-and-loop tape applicator without weight. In some embodiments, gel, hydrogel, or pressure sensitive adhesive may be used.

FIG. 17 illustrates an eighth embodiment of the of the HTR fracture applicator strap assembly. The eighth embodiment is a weighted version with retaining and alignment fixture in the center and varying weights and sizes. The HTR fracture applicator strap assembly 700 may include two pouches 710, 712 to hold weight. As an example, the weight may be in the form of a light, high volume material, such as plastic pellets. The HTR fracture applicator strap assembly 700 may include one or more arcuate portions 714 and may include an opening 716 to receive the retaining and alignment fixture 30. Adhesive can be added to the bottom to secure the applicator to the skin. Additionally, a strap and hook-and-loop tape may be used to hold the HTR fracture applicator strap assembly 700 in place.

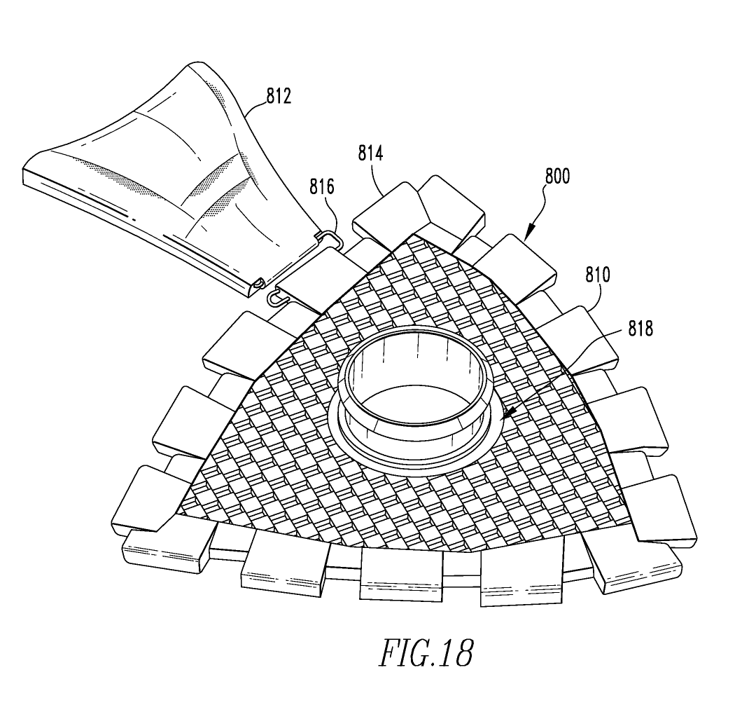

FIG. 18 illustrates a ninth embodiment of the of the HTR fracture applicator strap assembly. The HTR fracture applicator strap assembly 800 includes a pad 810 and at least one lobe 812. In some embodiments, the assembly 800 may include a plurality of lobes 812. The pad 810 includes a plurality of loops 814 and an opening 818 to receive the retaining and alignment fixture 30. The lobe 812 may be a weighted pouch or a piece of adhesive gel, such as hydrogel. The lobe 812 includes a hook 816, which can connect to the loops 814.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, while FIGS. 10A-10C illustrate edging and stitching, other structure and/or methods may be used to affix the assembly components together. Thus, the breadth and scope of the present invention should not be limited by any of the above-described exemplary embodiments, but should be defined only in accordance with the following claims appended hereto and their equivalents.

1. A hard-to-reach fracture applicator strap assembly, the assembly comprising:
a) a bottom portion;
b) a stiffener in contact with the bottom portion;
c) a large pouch portion in contact with the stiffener;
d) a small pouch portion in contact with the stiffener; and
e) a top portion in contact with at least one of the large pouch portion and the small pouch portion.

2. The hard-to-reach fracture applicator strap assembly of claim 1, wherein the bottom portion and the top portion have a shape selected from the group consisting of bow-tie, oval, rectangular, elliptical, lozenge, navicular, oblong, and triangular.

3. The hard-to-reach fracture applicator strap assembly of claim 1, wherein the bottom portion and the top portion are made of a material selected from the group consisting of polymers, synthetic fabrics, and natural fabrics.

4. The hard-to-reach fracture applicator strap assembly of claim 1, further comprising edging and stitching.

5. The hard-to-reach fracture applicator strap assembly of claim 1, wherein the large pouch portion and the small pouch portion are arranged to provide a space D around a perimeter of the stiffener, and the space D may range from about 0.1 inches to about 1 inch.

6. The hard-to-reach fracture applicator strap assembly of claim 6, wherein the large pouch portion and the small pouch portion each have one or more weighted compartments.

7. The hard-to-reach fracture applicator strap assembly of claim 6, wherein the weighted compartments each further comprise a weight selected from the group of metal pellets and plastic pellets.

8. The hard-to-reach fracture applicator strap assembly of claim 6, wherein the large pouch portion has four compartments and the small pouch portion has two compartments.

9. The hard-to-reach fracture applicator strap assembly of claim 1, wherein the bottom portion, the stiffener, and the top portion each include an opening.

10. The hard-to-reach fracture applicator strap assembly of claim 9, wherein the openings are co-axial when assembled.

11. The hard-to-reach fracture applicator strap assembly of claim 9, wherein the openings are shaped and dimensioned to receive a retaining and alignment fixture.

12. A method of applying ultrasound energy in hard to reach places, comprising:
strapping an ultrasound transducer to a patient using a fracture applicator assembly, the assembly including:
a) a bottom portion;
b) a stiffener in contact with the bottom portion;
c) a large pouch portion in contact with the stiffener;
d) a small pouch portion in contact with the stiffener; and
e) a top portion in contact with at least one of the large pouch portion and the small pouch portion.
